(19) United States
(12) Patent Application Publication
(10) Pub. No.: US 2014/0017165 A1
(43) Pub. Date: Jan. 16, 2014

(54) DNA REPAIR ENZYME INHIBITOR NANOPARTICLES AND USES THEREOF

(71) Applicants: Zhuang Wang, Chapel Hill, NC (US); Michael Edward Pacold, Chapel Hill, NC (US); Michael Edward Werner, Chapel Hill, NC (US); Shrirang Karve, Chapel Hill, NC (US)

(72) Inventors: Zhuang Wang, Chapel Hill, NC (US); Michael Edward Pacold, Chapel Hill, NC (US); Michael Edward Werner, Chapel Hill, NC (US); Shrirang Karve, Chapel Hill, NC (US)

(21) Appl. No.: 13/979,271

(22) PCT Filed: Jan. 11, 2012
(86) PCT No.: PCT/US12/20915

§ 371 Date: Sep. 30, 2013

Related U.S. Application Data
(60) Provisional application No. 61/431,689, filed on Jan. 11, 2011.

(51) Int. Cl. A61K 31/366; A61K 47/34; A61K 9/16

(52) U.S. Cl. CPC A61K 31/366; A61K 9/16; A61K 47/34
USPC 424/1.37; 424/490; 514/453; 424/9.6; 424/9.3; 424/9.44; 424/9.5

(57) ABSTRACT

This invention relates generally to the discovery of novel nanoparticles for delivery of DNA double-stranded break (DSB) repair enzyme inhibitors such as wortmannin or wortmannin analogues. In one embodiment, these nanoparticles comprise a polylactide polyglycolide (PLGA) copolymer and a polyethylene glycol (PEG). In addition methods of treatment and methods of enhancing radiation treatments are also provided.

### CROSS REFERENCE TO RELATED APPLICATIONS

This application claims the benefit of 61/431,689 filed Jan. 11, 2011, which is hereby incorporated by reference in its entirety.

### STATEMENT REGARDING FEDERALLY SPONSORED RESEARCH OR DEVELOPMENT

This invention was made in part with government support under grant number 5-K12-CA120780-01-05 awarded by the National Cancer Institute (NCI). The United States Government has certain rights in the invention.

### 1. FIELD OF THE INVENTION

This invention relates generally to the discovery of novel nanoparticles for delivery of DNA double-stranded break (DSB) repair enzyme inhibitors such as wortmannin or wortmannin analogues. In one embodiment, these nanoparticles comprise a polylactide polyglycolide (PLGA) copolymer and a polyethylene glycol (PEG).

### 2. BACKGROUND OF THE INVENTION

### 2.1. Introduction

Rapid advances in drug discovery and development have resulted in numerous innovative drugs with strong potential in therapies for life threatening diseases. Many small molecule drugs that have shown great promise as cancer therapeutics in preclinical studies but their clinical potentials were not demonstrated due to many shortcomings such as poor solubility, instability in vivo, high toxicity, and low bioavailability. Among such molecules are wortmannin and other DNA double-strand break (DSB) repair enzyme inhibitors which were thought to hold great promise as new cancer therapeutics.

### 2.2. Inhibitors of Repair Proteins of DNA Double Strand Breaks

DNA double-strand breaks (DSB) are breaks in the phosphate-deoxyribose backbone of both strands of the DNA double helix. DNA DSBs can result spontaneously or from cells' exposure to radiation and/or therapeutic agents that can induce DNA damage. If DSBs are not repaired, the result is severe genomic instability that generally leads to cell death. Therefore, the DSB repair mechanisms are critical in maintaining cell survival. There are a number of proteins that participate in the DNA DSB repair process, and they act to repair damages before critical cell cycle checkpoints are reached (Hoeijmakers, 2001 Nature 411: 366-374). In cancer treatment, it is long thought that these proteins involved in DNA DSB repair might be excellent therapeutic targets.

There are three key kinases involved in the detection and repair of DNA DSBs, Ataxia-telangiectasia mutated (ATM), ATM and Rad3 related (ATR), and DNA-dependent protein kinase (DNA-PK). All three of these proteins belong to a family of proteins called phosphatidylinositol 3-kinase related kinase (PIKKs). ATM and ATR signal to the cell cycle and apoptotic pathways that DNA DSBs have occurred (reviewed in Abraham, 2001 Genes Dev 15: 2177-2196). DNA-PK repairs DNA DSBs through a process called non-homologous end-joining (Mahaney et al. 2009 Biochem J. 417(3):639-50). In the last several decades, many inhibitors of DNA DSB repair proteins have been identified and characterized. Initially, phosphatidylinositol 3-kinase (PI3-K) inhibitors were found to also inhibit PIKKs, namely wortmannin and its analogue, LY294002 (Sarkaria et al. 1998 Cancer Res 58: 4375-4382) (Izzard et al. 1999 Cancer Res 59: 2581-2586). However, LY294002 has been used as a model to design more selective PIKK inhibitors. Hickson et al. (2004 Cancer Res 64: 9152-9159) developed 2-morpholin-4-yl-6-thianthren-1-yl-pyran-4-one (KU-55933), which inhibits ATM at low nanomolar concentrations. Hickson was also able to demonstrate KU-55933 is a chemosensitizer on cancer cell lines. Similar studies have produced NU7441, a potent and selective inhibitor for DNA-PK with an IC₅₀ of 13 nmol/L and inhibitory activity for other PIKKs at higher concentrations (Zhao et al. 2006 Cancer Res 66: 5354-5362). Also, small molecule drug screening has yielded SU11752, a DNA-PK inhibitor that is a more specific inhibitor of DNA-PK than wortmannin and demonstrated radiosensitization in a glioblastoma cell line (Ismail et al. 2004 Oncogene 23: 873-882).

Though DNA DSB repair inhibitors held high promise as chemotherapeutics and/or sensitizers to radiotherapy and chemotherapy and many inhibitors were promising in in vitro studies, these drugs could not be translated clinically due to drug delivery challenges. Most of the members of this class of therapeutics are poorly soluble in water. Moreover, these inhibitors are so effective at inhibiting DNA DSB that their potential systemic toxicity is prohibitive to their translation.

### 2.3. Chemistry and Biology of Wortmannin

More than 50 years ago the naturally occurring fungal metabolite, wortmannin, was isolated and purified from culture broths of the fungus Penicillium wortmanni (Brian et al., 1957, Trans Brit Mycol Soc 40: 365). In 1972 the crystal structure and absolute stereochemistry were reported. Other fungal sources include Talaromyces wortmannin (Nakanishi et al., 1992, J Biol Chem, 267 (4): 2157-2163); Myrothecium roridium and Fusarium oxysporum (Abbas et al., 1988, Appl. Environ. Microbiol, 54(5): 1267-1274); or the American Type Culture Collection (ATCC). Shibbasaki et al. has reported the total synthesis of wortmannin and Broka and Ruhland have reported synthesis of the core structure, Sato et al., 1996 Tet Lett 73: 6144-6144; Mizutani et al., 2002 Angew Chem Int Ed 43: 4680-4681; Broka & Ruhland, 1992 J Org Chem 57: 4888-4894. For a review, Wifp & Halter, 2005 RSC Org Biomol Chem 3: 2053-2061.

The structure for wortmannin is shown below, WMT 1.

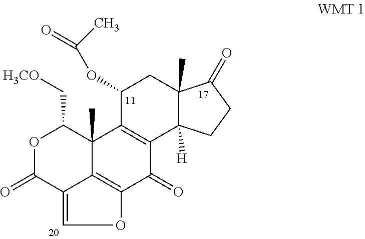 [SMILES: COC[C@H]1OC(=O)c2coc3c2[C@@]1(C)C1=C(C3=O)[C@@H]2CCC(=O)[C@@]2(C)C[C@H]1OC(C)=O]

In 1994, wortmannin was reported to be a selective and potent inhibitor of phosphotidylinositol-3-kinase (PI-3-kinase or PI3K) (Powis et al., 1994, Cancer Res, 54, 2419-2423). PI3Ks are a family of enzymes involved in cellular functions such as cell growth, proliferation, differentiation, motility, survival and intracellular trafficking, which in turn are involved in cancer. PI3Ks also play a key role in insulin signaling and diabetes. The mechanism of action was studied by Wymann who reported in 1996 that wortmannin covalently inactivates PI3K when Lys-802 forms a vinylogous carbamate with the C20 on furan ring of wortmannin (Wymann et al., 1996 Mol Cell Bio 16(4):1722-1733). Wortmannin analogues and their use in inhibiting PI-3-kinase activity as well as tumor formation are described in U.S. Pat. Nos. 5,378,725 (Bonjouklian et al.), 7,335,679 (Powis et al.), and 7,776,908 (Powis et al.). Stereochemical wortmannin derivatives are described in U.S. Pat. No. 5,480,906, European Patent 0762879B1 (Creemer et al.).

As mentioned above, specific inhibitors of PI3Ks, DNA DSB repair inhibitors like wortmannin, created a great deal of interest from the pharmaceutical industry many years ago. See for example, Stein et al., 2000, Mol Med Today 6(9) 347-57, “PI3-kinase inhibition: a target for drug development?” In spite of the promise, little or no clinically relevant results have been reported. Amongst of the problems associated with the development of wortmannin as a therapeutic are its poor solubility in water, half-life of approximately 10 minutes in serum, limited bioavailability, and high toxicity. Even in low dosages, wortmannin in pure form is often lethal to laboratory animals. Nonetheless, wortmannin is often used as a research reagent to study DNA repair, receptor-mediated endocytosis, and cell proliferation and is commercially available.

Recently, Bae et al. reported the synthesis and biological activity of wortmannin covalently-linked onto poly(ethylene glycol)-poly(aspartate hydrazide) block copolymers alone or in combination with doxorubicin conjugates. In these studies, wortmannin and the wortmannin conjugates (CMM-DW100) showed unexpectedly low cytotoxicity against cancer cells. See FIG. 6, Bae et al., 2007 J Controlled Rel 122: 324-330.

The delivery of pharmaceuticals with poor water solubility (hydrophobic) is problematic as most of the body compartments, including the blood circulation and intracellular fluids are an aqueous environment. As a result, the direct injection of hydrophobic therapeutic agents often results in harmful side effects due to hypersensitivity, hemolysis, cardiac, and neurological symptoms. Moreover, in vivo wortmannin reacts with free lysines limiting its bioavailability.

The present invention is directed to the unexpected result that encapsulated DNA DSB repair inhibitors such as wortmannin in nanoparticles (NPs) are highly bioavailable. These nanoparticles provide a delivery system for DNA DSB repair inhibitors which improve their solubility, increase their stability, increase their bioavailability, and lower their toxicity. These nanoparticles have shown synergistic effects as radiation sensitizers.

### 3. SUMMARY OF THE INVENTION

In particular non-limiting embodiments, the present invention provides a nanoparticle comprising (i) a polylactide polyglycolide (PLGA) copolymer, (ii) a polyethylene glycol (PEG), and (iii) a DNA double-stranded break (DSB) repair enzyme inhibitor. The nanoparticle may be a poly(lactide-co-glycolide) copolymer with a lactic acid to glycolic acid monomer ratio from about 10:90 to about 90:10; about 20:80 to about 80:20; about 30:70 to about 70:30; about 40:60 to about 60:40; or about 50:50. The nanoparticle may further comprise a lipid. Alternatively, the nanoparticle may consisting essentially of (i) the polylactide polyglycolide (PLGA) copolymer, (ii) the polyethylene glycol (PEG), (iii) the DNA double-stranded break (DSB) repair enzyme inhibitor, and the lipid. In one embodiment, the polylactide (PLA) is a polylactide polyglycolide (PLGA) copolymer, the polylactide is coated with the lipid, and the lipid is coated with the polyethylene glycol (PEG). In some embodiments, the DNA double-stranded break (DSB) repair enzyme inhibitor is wortmannin or a wortmannin analogue, KU-55933, LY294002, NU7441 or SU11752 which may or may not homogenously dissolved in the polylactide polyglycolide (PLGA) copolymer.

In some embodiments, the nanoparticle may further comprise a targeting moiety which may be a carbohydrate, a fatty acid, a glycopeptide, a glycoprotein, a lipid, a peptide, a polymer, a polynucleotide, a protein, or a small molecule. In some embodiments, the targeting moiety is a folic acid analogue, an antibody or an antibody fragment, or an aptamer.

In some embodiments, the nanoparticle is about 1 nm to about 1000 nm in at least one dimension; about 10 nm to about 500 nm in at least one dimension; about 20 nm to about 100 nm in at least one dimension; or about 50 nm to about 80 nm in at least one dimension. The nanoparticle may have a zeta potential between about +100 mV and about −100 mV; about −0 mV and about −60 mV; about −20 mV and about −50 mV; or about −35 mV and about −45 mV.

The nanoparticle may further comprise an imaging agent which may be a chemiluminescent agent, a colorimetric agent, an enzyme, a fluorophore, a light emitting agent, a light scattering agent, a magnetic resonance imaging (MRI) contrast agent, a positron emission tomography (PET) imaging agent, a radioisotope, or an ultrasound imaging agent.

The nanoparticle may comprise about 0.1% to about 90% wortmannin or a wortmannin analogue by weight. It may also comprise about 1% to about 60%; about 2% to about 50%; about 3% to about 40%; or about 4% to about 30% wortmannin or a wortmannin analogue by weight.

In one embodiment, a method of treating a phosphoinositide 3-kinase associated disorder in a mammal is provided which comprises administering to the mammal a therapeutically effective amount of a nanoparticle comprising (i) a polylactide polyglycolide (PLGA) copolymer, (ii) a polyethylene glycol (PEG), and (iii) a DNA double-stranded break (DSB) repair enzyme inhibitor. The phosphoinositide 3-kinase associated disorder may be an inflammatory, an immunological, a metabolic, or a proliferative disorder. The immunological disorder may be associated with organ transplantation or an autoimmune disorder. The metabolic disorder may be diabetes or an insulin-related disorder. The proliferative disorder may be a cancer, such as breast cancer, chronic myeloid leukemia, colon cancer, head and neck cancer, glioblastoma, lung cancer, nasopharyngeal cancer, ovarian cancer, or prostate cancer.

For treatment of cancer, the method may further comprise administering an alkylating agent, an anticancer antibiotic, an antimetabolite, a hormone, a monoclonal antibody, or a plant alkyloid.

In another embodiment, a method of enhancing radiation treatment in a mammal comprising administering to the mammal a therapeutically effective amount of a nanoparticle comprising (i) a polylactide polyglycolide (PLGA) copolymer, (ii) a polyethylene glycol (PEG), and (iii) a DNA double-stranded break (DSB) repair enzyme inhibitor is provided.

### 4. BRIEF DESCRIPTION OF THE FIGURES

FIG. 1 shows a schematic of an exemplary particle with a PLGA core with encapsulated wortmannin.

FIG. 2 shows wortmannin release from nanoparticles in PBS at 37 degrees. Particles were loaded with wortmannin at 10% or 20% by weight. The gray circles are 10%; the black circles are 20% by weight.

FIG. 3A, FIG. 3B and FIG. 3C show the radiosensitization effect of wortmannin encapsulated nanoparticles on three different cancer cell lines by different delivery methods. FIG. 3A shows data for KB cells; FIG. 3B shows data for PC-3 cells; and FIG. 3C shows data for HT29 cells.

FIG. 4 shows wortmannin nanoparticle inhibits phosphorylation of DNA-PK after radiation.

FIG. 5 shows in vivo antitumor activity of wortmannin nanoparticles in combination with radiation.

FIG. 6 shows the biodistribution of nanoparticles in tumor bearing mice.

FIG. 7 shows wortmannin chemosensitization of lung cancer cell line H460.

FIG. 8 shows radiosensitization with KU55933 of lung cancer line A549.

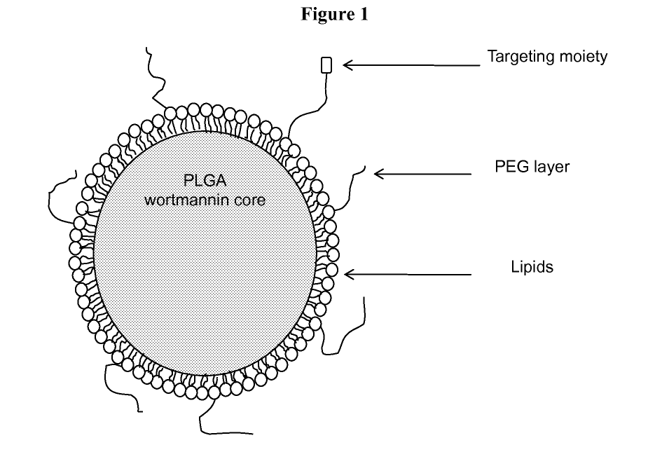

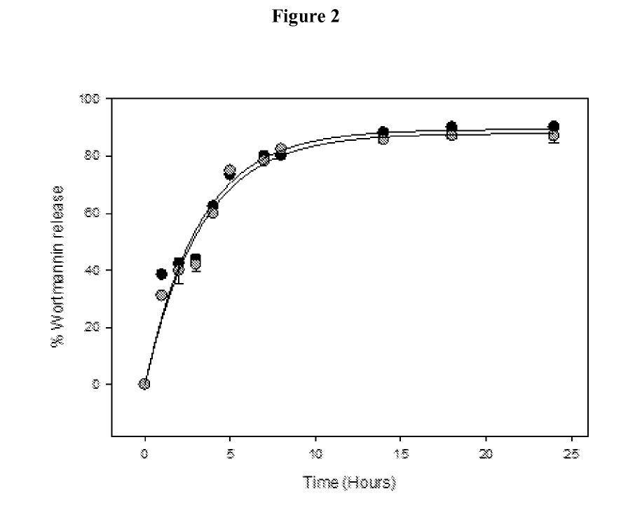

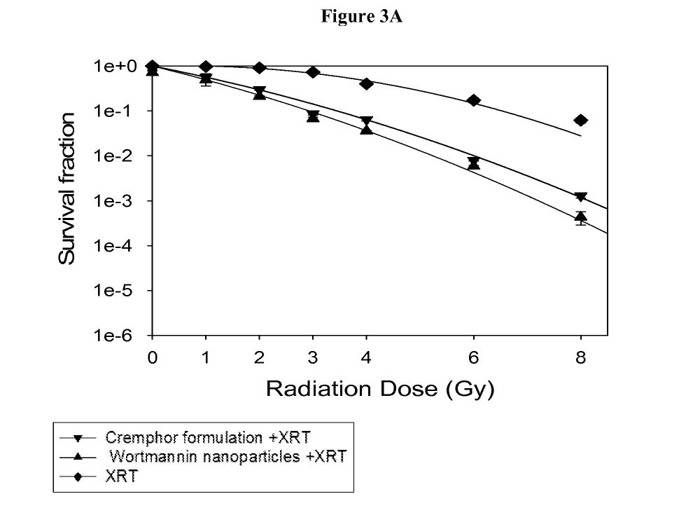

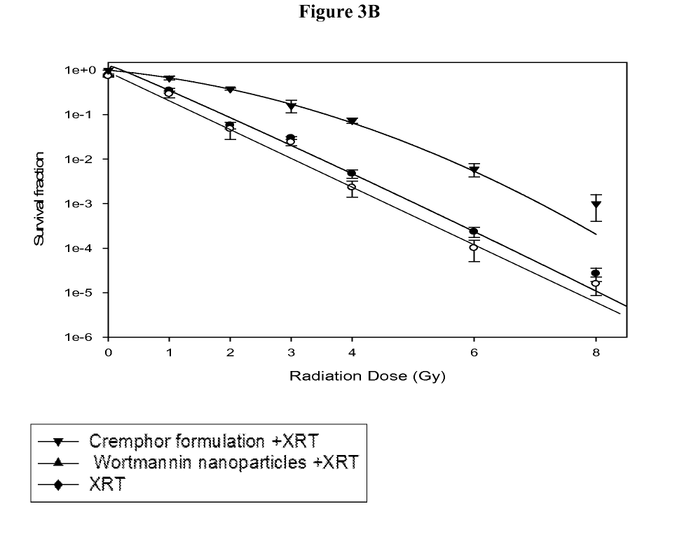

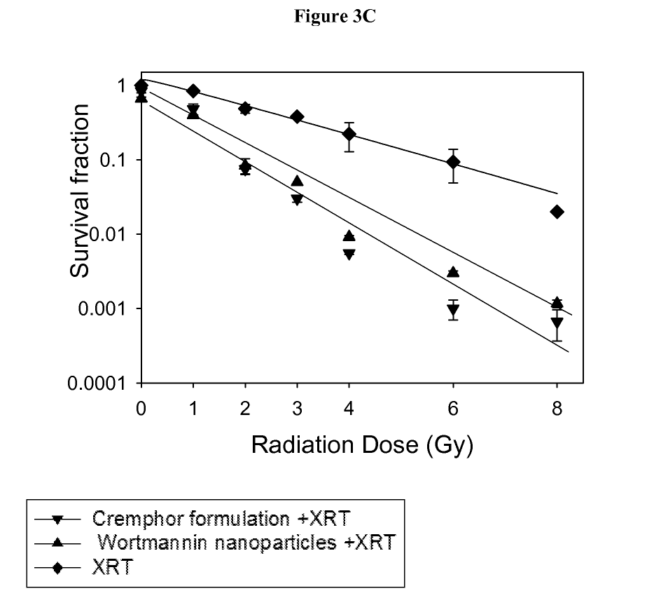

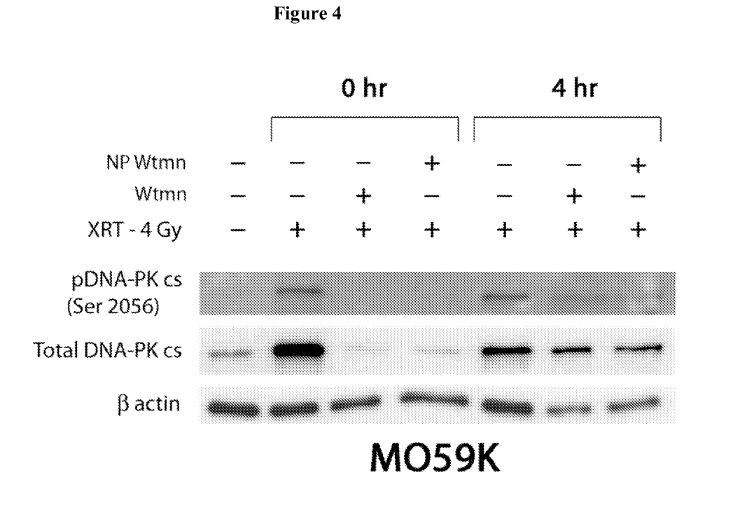

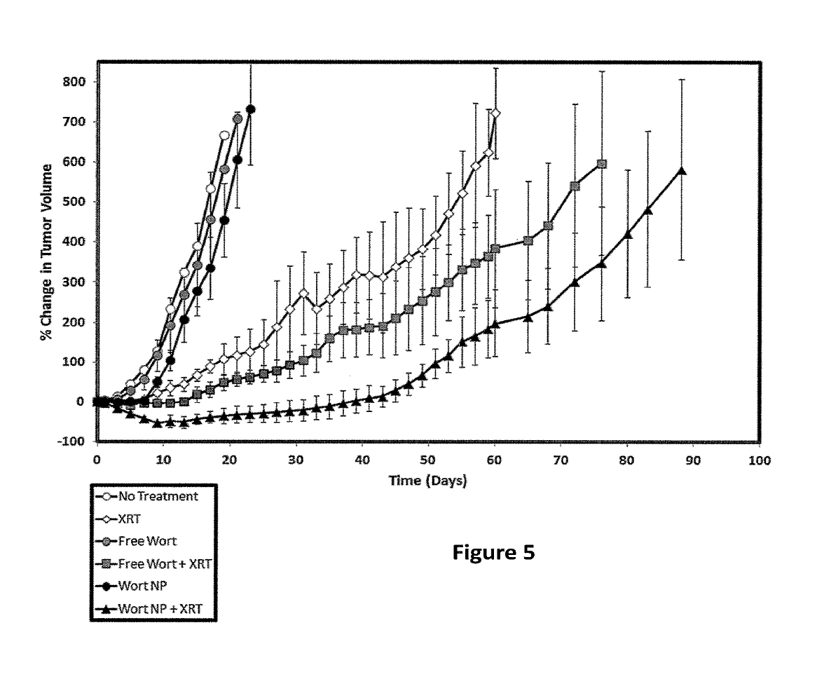

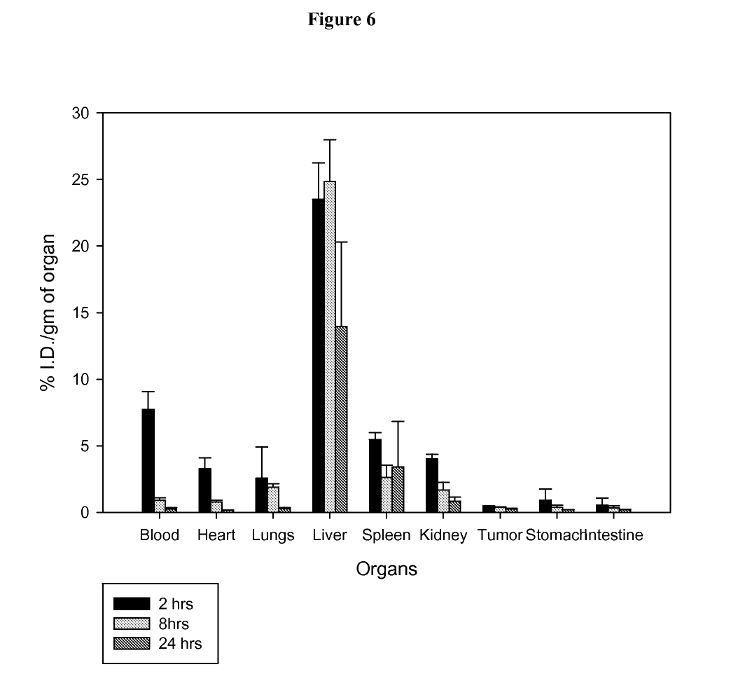

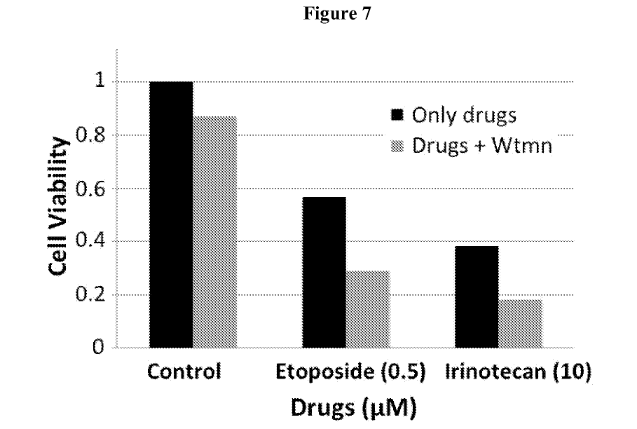

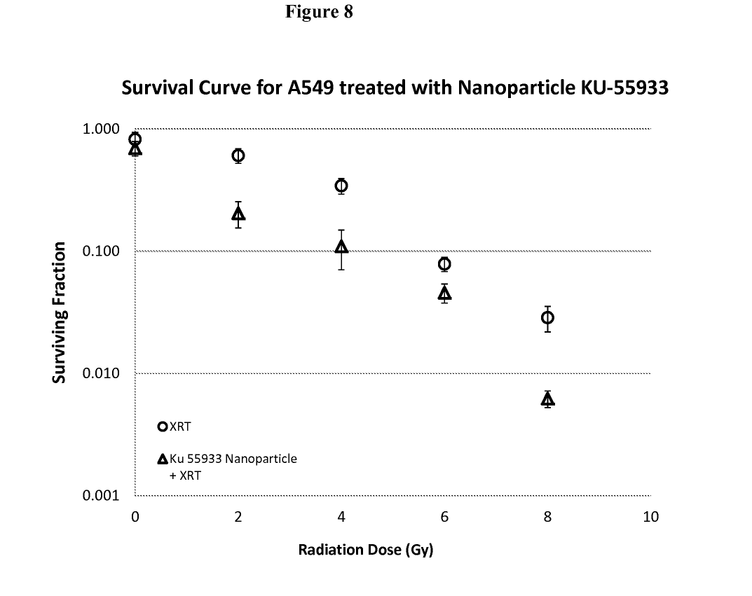

### 5. DETAILED DESCRIPTION OF THE INVENTION

### 5.1. Introduction

This invention pertains to formulations of DNA double-stranded break (DSB) repair enzyme inhibitors such as wortmannin and KU-55933 that show unexpected increases in bioavailability and synergistic effects when used in combination with radiation. Furthermore, the invention provides nanoparticle formulations of DNA double-stranded break (DSB) repair enzyme inhibitors suitable for use in vivo, formulations of DNA double-stranded break (DSB) repair enzyme inhibitors in nanoparticles in a pharmaceutically acceptable carrier. The present invention provides for a method of inhibiting PI-3-kinase activity as well as other targets of DNA double-stranded break (DSB) repair enzyme inhibitors by administration of an effective amount of DNA double-stranded break (DSB) repair enzyme inhibitors encapsulated in nanoparticles. The DNA double-strand break repair enzyme inhibitor may be a covalent inhibitor, e.g., wortmannin. It may be a competitive inhibitor, e.g., KU-55933. It may be a non-competitive inhibitor. It may be a DNA-dependent protein kinase (DNA-PK) inhibitor such as NU7441 or IC87361 (Shinohara et al. 2005 Cancer Res 65: 4987-4992).

The present invention also provides a drug delivery system for delivering other agents (e.g., therapeutic agent, diagnostic agent, or prophylactic agent) in combination with nanoparticle formulation of a DNA double-stranded break (DSB) repair enzyme inhibitor such as wortmannin or a wortmannin analogue. The system may be further modified for selectively delivering the combination of agents to particular organs, tissues, cells, and/or intracellular compartments. In certain embodiments, the agents are specifically delivered to diseased tissues. In certain specific embodiments, the agents are specifically delivered to tumors (e.g., malignant tumors or benign tumors). In certain specific embodiments, the agents are specifically delivered to cells (e.g., cells of the immune system). The delivery system may also be used in diagnosis or imaging. In certain embodiments, agents for treatment as well as diagnosis are provided in the same drug delivery device.

### 5.2. Definitions

Unless defined otherwise, all technical and scientific terms used herein have the same meaning as is commonly understood by one of ordinary skill in the art. All patents, applications, published applications and other publications are incorporated by reference in their entirety. In the event that there are more than one definition for a term herein, those in this section prevail unless stated otherwise.

“Alkyl” refers to a straight or branched hydrocarbon chain group consisting solely of carbon and hydrogen atoms, containing no unsaturation, having from 1 to 16, 1 to 8, 1 to 6 or 1 to 4 carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), etc.

“Alkenyl” refers to a straight or branched hydrocarbon chain group consisting solely of carbon and hydrogen atoms, containing at least one double bond, in certain embodiment, having from 2 to 10 carbon atoms, from 2 to 8 carbon atoms, or from 2 to 6 carbon atoms, and which is attached to the rest of the molecule by a single bond or a double bond, e.g., ethenyl, prop-1-enyl, but-1-enyl, pent-1-enyl, penta-1,4-dienyl, etc.

“Alkynyl” refers to a straight or branched hydrocarbon chain group consisting solely of carbon and hydrogen atoms, containing at least one triple bond, having from two to ten carbon atoms, and which is attached to the rest of the molecule by a single bond or a triple bond, e.g., ethynyl, prop-1-ynyl, but-1-ynyl, pent-1-ynyl, pent-3-ynyl, etc.

“Acyl” refers to the group having the formula —C(O)R wherein R is alkyl or haloalkyl, where the alkyl may be optionally substituted by one or more substituents, in one embodiment, one, two or three substituents independently selected from the group consisting of nitro, halo, hydroxyl, alkoxy, oxo, thioxo, amino, carbony, carboxy, azido, cyano, cycloalkyl, heteroaryl, and heterocyclyl.

“Alkoxy” refers to the group having the formula —OR wherein R is alkyl or haloalkyl, where the alkyl may be optionally substituted by one or more substituents, in one embodiment, one, two or three substituents independently selected from the group consisting of nitro, halo, hydroxyl, alkoxy, oxo, thioxo, amino, carbony, carboxy, azido, cyano, cycloalkyl, heteroaryl, and heterocyclyl.

“Anti-cancer agents” refers to anti-metabolites (e.g., 5-fluorouracil, methotrexate, fluoroarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxrubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent® and Bevacizumab), an EGFR inhibitor (e.g., cetuximab, erlotinib, gefitinib), or any other cytotoxic agents, (estramustine phosphate, prednimustine), hormones or hormone agonists, antagonists, partial agonists or partial antagonists, kinase inhibitors, and radiation treatment.

“Aryl” refers to a group of carbocylic ring system, including monocyclic, bicyclic, tricyclic, tetracyclic C₆-C₁₈ ring systems, wherein at least one of the rings is aromatic. The aryl may be fully aromatic, examples of which are phenyl, naphthyl, anthracenyl, acenaphthylenyl, azulenyl, fluorenyl, indenyl and pyrenyl. The aryl may also contain an aromatic ring in combination with a non-aromatic ring, examples of which are acenaphene, indene, and fluorene. The term includes both substituted and unsubstituted moieties. The aryl group can be substituted with any described moiety, including, but not limited to, one or more moieties selected from the group consisting of halo (fluoro, chloro, bromo or iodo), alkyl, hydroxyl, amino, alkoxy, aryloxy, nitro and cyano.

“Cycloalkyl” refers to a stable monovalent monocyclic or bicyclic hydrocarbon group consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl, norbornane, norbornene, adamantyl, bicyclo[2.2.2]octane and the like.

“Haloalkyl” refers to a straight or branched hydrocarbon chain group, containing no unsaturation, having from 1 to 16, 1 to 8, 1 to 6 or 1 to 4 carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), etc. The term includes both substituted and unsubstituted moieties. The alkyl group can be substituted with any described moiety, including, but not limited to, one or more moieties selected from the group consisting of halo (fluoro, chloro, bromo or iodo).

“Pharmaceutically acceptable salts” include, but are not limited to, salts of mineral acids, such as hydrochlorides; and salts of organic acids, such as but not limited to mesylate, esylate, tosylate, besylate, brosylate, camphorsulfonate, hydrobromide, phosphate, sulfate, trifluoroacetate, acetate, benzoate, fumarate, malate, maleate, oxalate, succinate and tartrate.

“Subjects” include, but are not limited to, humans, mammals, animals such as dogs, cats, farm animals, laboratory animals such as rats, mice, monkeys.

### 5.3. Wortmannin and Wortmannin Analogues

In one aspect, the drug delivery system is a particle (e.g., picoparticle, nanoparticle, or microparticle) containing wortmannin or wortmannin analogues. The structure of wortmannin (WTM 1) is shown below

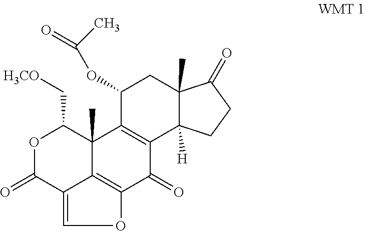 [SMILES: COC[C@H]1OC(=O)c2coc3c2[C@@]1(C)C1=C(C3=O)[C@@H]2CCC(=O)[C@@]2(C)C[C@H]1OC(C)=O]

In other embodiments, the structure is a wortmannin analogue such as the structures below.

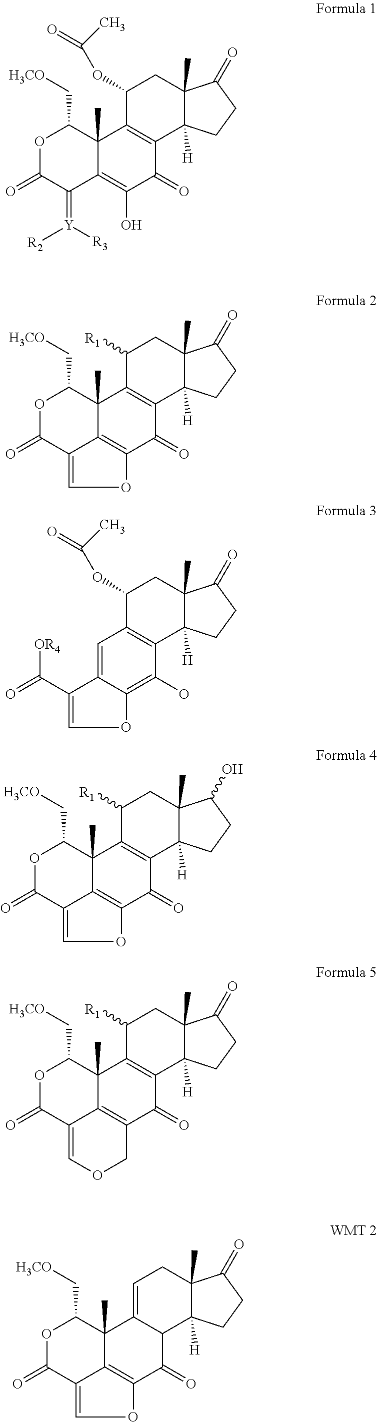

The wortmannin analogues, include stereoisomers at any of the chiral centers. Wortmannin analogues include compounds such as WMT 2, 11-desacetoxy-Δ9-wortmannin. The semi-synthetic preparation and characterization of a variety of wortmannin analogues (Formulas 1-5) are reported in Wipf & Halter, 2005, supra; Norman et al., 1995 Bioorg Med Chem Lett 5: 1183-1186; Dodge et al., 1995 Bioorg Med Chem Lett 5: 1713-1718; Norman et al., 1996 J Med Chem 39: 1106-1111; Creemer et al., 1996 J Med Chem 39: 5021-5024; U.S. Pat. Nos. 5,378,725; 5,480,906; 7,335,679; and 7,776,908, the contents of which are hereby incorporated by reference in their entirety. In one embodiment, R₁ is H, OH, acyl, alkenyl, alkyl, alkynyl, aryl, cycloalkyl, or haloalkyl. Y is a heteroatom. R2 and R3 are independently acyl, alkenyl, alkyl, alkynyl, aryl, cycloalkyl, or haloalkyl. R4 is alkyl, aryl, cycloalkyl, or haloalkyl. Non-limiting examples of specific wortmannin analogues are shown in Table 1.

TABLE 1
Wortmannin and wortmannin analogues

| Compound | | | | | | |
|---|---|---|---|---|---|---|
| Name | Formula | R1 | R2 | R3 | R4 | Reference/source |
| WMT 1 | n/a | n/a | n/a | n/a | n/a | Sigma-Aldrich |
| WMT 2 | n/a | n/a | n/a | n/a | n/a | Haeflinger, |
| | | | | | | Helv. Chem Act |
| | | | | | | 56(8) 2901 |
| WMT 3 | #1 Y = N | n/a | —C₃H₅ | —C₃H₅ | n/a | 7,335,679 |
| WMT 4 | #1 Y = N | n/a | —C₂H₄— | —C₂H₄— | n/a | 7,335,679 |
| WMT 5 | #1 Y = N | n/a | -Bzyl | H | n/a | 7,335,679 |
| WMT 6 | #1 Y = S | n/a | CH₃ | CH₃ | n/a | 7,335,679 |
| WMT 7 | #2 | OH | n/a | n/a | n/a | 5,480,906 |
| WMT 8 | #2 | OCOC₃H₇ | n/a | n/a | n/a | 5,480,906 |
| WMT 9 | #2 | OCOC₄H₉ | n/a | n/a | n/a | 5,480,906 |
| WMT 10 | #2 | OCOBzyl | n/a | n/a | n/a | 5,480,906 |

### 5.4. Polymeric Particles

In certain embodiments, the particle is a polymeric particle. In certain embodiments, the particle comprises a polymeric core with a shell coating the core. In certain embodiments, the particle comprises a polymeric core coated with a lipid (e.g., a lipid monolayer or lipid bilayer). In certain embodiments, the particle is a liposome. In certain embodiments, the particle is a micelle. One or both of the agents to be delivered may be inside the particle (e.g., in the core), in the shell or coating portion of the particle, or associated with the surface of the particle.

Any of the above embodiments may also include a targeting agent (e.g., aptamers, antibodies, antibody fragments, etc.) on the surface of the particle. In general, the cell to be targeted by the inventive particle includes a target which is specifically bound by the targeting agent. The agents are able to be delivered to the particular targeted organ, tissue, cell, extracellular matrix, extracellular compartment, and/or intracellular compartment once the targeting agent specifically binds to the target on the cell or intracellular compartment.

The whole particle or a portion of the inventive particle may be biodegradable. In certain embodiments, the entire particle is biodegradable. In other embodiments, only a portion of the particle is biodegradable (e.g., the outer layer of the particle). In general, a biodegradable substance is one that can be broken down under physiological conditions. In certain embodiments, the components of the inventive particles are biocompatible. That is, the materials used to prepare the particles do not lead to an adverse reaction when introduced into a living biological system.

In certain embodiments, an inventive particle is any entity that measures less than 500 microns (μm) in at least one dimension (e.g., diameter or length). In some embodiments, inventive particles measure less than 300 μm in at least one dimension. In some embodiments, inventive particles measure less than 200 μm, less than 100 μm, less than 75 μm, less than 50 μm, or less than 10 μm. In some embodiments, inventive particles are nanoparticles that measure less than 1000 nanometers (nm) in at least one dimension. In some embodiments, nanoparticles measure less than 900 nm, 800 nm, 700 nm, 600 nm, 500 nm, 400 nm, 300 nm, 200 nm, or 100 nm. In some embodiments, the nanoparticles measure less than 900 nm, 800 nm, 700 nm, 600 nm, 500 nm, 400 nm, 300 nm, 200 nm, or 100 nm in at least one dimension. In other embodiments, the nanoparticle is about 1 nm to about 1000 nm in at least one dimension. In alternative embodiments, the nanoparticle is about 10 nm to about 500 nm in at least one dimension. In some embodiments, the nanoparticle is about 20 nm to about 100 nm in at least one dimension. In other embodiments, the nanoparticle is about 50 nm to about 80 nm in at least one dimension.

In one embodiment, the nanoparticles release 50% of the wortmannin or analogue into solution at 37% in 15 minutes. In another embodiment, the nanoparticles release 50% in 30 minutes; 1 hour; 2 hours; 5 hours; 10 hours; or 20 hours. In some embodiments, the nanoparticles release 50% of the wortmannin or analogue in 1 to 5 hours or 2 to 4 hours.

In some embodiments, the particles are microparticles (e.g., microspheres). In some embodiments, the particles are nanoparticles (e.g., nanospheres). In some embodiments, the particles are picoparticles (e.g., picospheres). In some embodiments, the particles are coated polymeric particles. In some embodiments, the particles are liposomes. In some embodiments, the particles are micelles.

Particles can be solid or hollow. The particles can comprise one or more layers (e.g., nanoshells, nanorings). The particles can be coated. In certain embodiments, the particles include an outer lipid monolayer. In certain embodiments, the particles include an outer lipid bilayer. In certain embodiments, the particles include a polymeric outer layer.

5.4.1. Specific Polymeric Matrices

In certain embodiments, the inventive particle comprises a polymer. In some embodiments, the DNA double-stranded break (DSB) repair enzyme inhibitor to be delivered and/or targeting moiety can be associated with the surface of, encapsulated within, surrounded by, and/or dispersed throughout a polymeric matrix. In certain embodiments, the inventive particle includes a polymeric core. In certain embodiments, the polymer used in the particle may comprise a natural or a semisynthetic polymer. Examples of such polymers include albumins, aliginic acids, carboxymethylcelluloses, sodium salt cross-linked, celluloses, cellulose acetates, cellulose acetate butyrates, cellulose acetate phthalates, cellulose acetate trimelliates, chitins, chitosans, collagens, dextrins, ethylcelluloses, gelatins, guargums, hydroxypropylmethyl celluloses (HPC), karana gums, methyl celluloses, poloxamers, polysaccharides, sodium starch glycolates, starch thermally modifieds, tragacanth gums, or xanthangums.polysaccharides,

In other embodiments, the polymer may be a synthetic polymer. Examples of synthetic polymers include cellophane (polyethylene-coated), monomethoxypolyethylene glycols (mPEG), nylons, polyacetals, polyacrylates, poly(alkylene oxides), polyamides, polyamines, polyanhydrides, polyargines, polybutylene oxides (PBO), polybutyolactones, polycaprolactones (PCL), polycarbonates, polycyanoacrylates, poly(dioxanones) (PDO), polyesters, polyethers, polyethylenes, poly(ethylene-propylene) copolymers, poly(ethylene glycols) (PEG), poly(ethylene imines), polyethylene oxides (PEO), polyglycolides (PGA), polyhydroxyacids, polylactides (PLA), polylysines, polymethacrylates (PMA), poly(methyl vinyl ethers) (PMV), poly(N-vinylpyrrolidinones) (NVP), polyornithines, poly(orthoesters) (POE), polyphosphazenes, polypropiolactones, polypropylenes, poly(propylene glycols) (PPG), polypropylene oxides (PPO), polypropylfumerates, polyserines, polystyrenes, polyureas, polyurethanes, polyvinyl alcohols (PVA), poly(vinyl chlorides) (PVC), poly (vinyl pyrrolidines) or silicon rubbers.

The polymer may be a homopolymer, a copolymer, a block copolymer with monomers from one or more the polymers above. If the polymer comprises asymmetric monomers, it may be regio-regular, isotactic or syndiotactic (alternating); or region-random, atactic. If the polymer comprises chiral monomers, the polymer may be stereo-regular or a racemic mixture, e.g., poly(D-, L-lactic acid). It may be a random copolymer, an alternating copolymer, a periodic copolymer, e.g., repeating units with a formula such as [AₙBₘ]. The polymer may be a linear polymer, a ring polymer, a branched polymer, e.g., a dendrimer. The polymer may or may not be cross-linked. The polymer may be a block copolymer comprising a hydrophilic block polymer and a hydrophobic block polymer.

The polymer may comprise derivatives of individual monomers chemically modified with substituents, including without limitation, alkylation, e.g., (poly C₁-C₁₆ alkyl methacrylate), amidation, esterification, ether, or salt formation. The polymer may also be modified by specific covalent attachments the backbone (main chain modification) or ends of the polymer (end group modifications). Examples of such modifications include attaching PEG (PEGylation) or albumin.

In certain embodiments, the polymer may be a poly(dioxanone). The poly(dioxanone) may be poly(p-dioxanone), see U.S. Pat. Nos. 4,052,988; 4,643,191; 5,080,665; and 5,019,094, the contents of which are hereby incorporated by reference in their entirety. The polymer may be a copolymer of poly(alkylene oxide) and poly(p-dioxanone), such as a block copolymer of poly(ethylene glycol) (PEG) and poly(p-dioxanone) which may or may not include PLA, see U.S. Pat. No. 6,599,519, the content of which is hereby incorporated by reference in its entirety.

The polymer used in the particle is a polyester, a polyester-polycation copolymer, or a polyester-polysugar copolymer. See U.S. Pat. No. 6,410,057, the content of which is hereby incorporated by reference in its entirety.

In some embodiments, the polymeric matrix may be a polyethylene oxide (POE). Examples of POE block copolymers include U.S. Pat. Nos. 5,612,052 and 5,702,717, the contents of which are hereby incorporated by reference in their entirety. In some embodiments, a polymeric matrix may be a polylactide (PLA), including poly(L-lactic acid), poly(D-lactic acid), poly(D-,L-lactic acid); a polyglycolide (PGA); poly(lactic-co-glycolic acid) (PLGA); poly(lactic-co-dioxanone) (PLDO) which may or may not include polyethylene glycol (PEG). See U.S. Pat. Nos. 4,862,168; 4,452,973; 4,716,203; 4,942,035; 5,384,333; 5,449,513; 5,476,909; 5,510,103; 5,543,158; 5,548,035; 5,683,723; 5,702,717; 6,616,941 (e.g., Table 1); 6,916,788 (e.g., Table 4, PLA-PEG, PLDO-PEG, PLGA-PEG), 7,217,770 (PEG-PLA); 7,311,901 (amphophilic copolymers); 7,550,157 (mPEG-PCL, mPEG-PLA, mPEG-PLDO, mPEG-PLGA, and micelles); U.S. Pat. Pub. No. 2010/0008998 (Table 2, PEG2000/4000/10,000-mPEG-PLA); PCT Pub. Nos. 2009/084801 (mPEG-PLA and mPEG-PLGA micelles), the contents of which are hereby incorporated by reference in their entirety. In some embodiments, a polymeric matrix can comprise proteins, lipids, surfactants, carbohydrates, small molecules, and/or polynucleotides.

In some embodiments, particles can be non-polymeric particles (e.g., metal particles, quantum dots, ceramics, inorganic materials, bone, etc.). In some embodiments, wortmannin to be delivered and/or targeting moiety can be covalently associated with a non-polymeric particle. In some embodiments, one or both of the agents to be delivered and/or targeting moiety is non-covalently associated with a non-polymeric particle.

5.4.2. Specific Lipids

Lipids may be (1) uncharged lipid components, e.g., cholesterol, ceramide, diacylglycerol, acyl(poly ethers) or alkylpoly(ethers); (2) neutral phospholipids, e.g., diacylphosphatidylcholines, sphingomyelins, and diacylphosphatidylethanolamines, (3) anionic lipids, e.g., diacylphosphatidylserine, diacylphosphatidylglycerol, diacylphosphatidate, cardiolipin, diacylphosphatidylinositol, diacylglycerolhemisuccinate, diaclyglycerolhemigluratate, cholesterylhemi succinate, cholesterylhemiglutarate, and the like; (4) polymer-conjugated lipids, e.g., N-[methoxy-(poly(ethylene glycol)diacylphosphatidylethanolamine, poly(ethylene glycol)-diacylglycerol, poly(ethylene glycol)ceramide; and (5) cationic lipids, e.g., 1,2-diacyl-3-trimethyl ammonium-propane (DOTAP), dimethyldioctadecylammonium bromide (DDAB), and 1,2-diacyl-sn-glycero 3-ethylphosphocholine. Monoacyl-substituted derivatives of these lipids, as well as di- and monoalkyl-analogs also included.

In certain embodiments, the lipid in the nanoparticle may be lecithin. Examples of lecithins include natural lecithin, a hydrogenated natural lecithin, a synthetic lecithin, 1,2-distearoyl-lecithin, dipalmitoyllecithin, dimyristoyllecithin, dioleolyllecithin, 1-stearoyl-2-oleoyllecithin, or 1-palmitoyl-2-oleoyllecithin, phosphatidyl choline, phosphatidyl inositol, phosphatidyl ethanolamine, and phosphatidic acid.

The lipid may be a lysophosphatidylcholine such as 1-myristoyl-sn-glycero-3-phosphocholine, 1-palmitoyl-sn-glycero-3-phosphocholine; a phosphatidic acid such as 1-stearoyl-sn-glycero-3-phosphocholine, 1,2-dierucoyl-sn-glycero-3-phosphate (sodium salt), 1,2-dilauroyl-sn-glycero-3-phosphate (sodium salt), 1,2-dimyristoyl-sn-glycero-3-phosphate (sodium salt), 1,2-dioleoyl-sn-glycero-3-phosphate (sodium salt), 1,2-dipalmitoyl-sn-glycero-3-phosphate (sodium salt), 1,2-distearoyl-sn-glycero-3-phosphate (sodium salt), 1,2-didecanoyl-sn-glycero-3-phosphocholine, 1,2-dierucoyl-sn-glycero-3-phosphocholine, 1,2-dilinoleoyl-sn-glycero-3-phosphocholine, 1,2-dilauroyl-sn-glycero-3-phosphocholine, 1,2-dimyristoyl-sn-glycero-3-phosphocholine, 1,2-dioleoyl-sn-glycero-3-phosphocholine, 1,2-dipalmitoyl-sn-glycero-3-phosphocholine, 1,2-distearoyl-sn-glycero-3-phosphocholine, egg-phosphotidyl choline (PC), hydrogenated egg PC, high purity hydrogenated soy PC, hydrogenated soy PC, 1-myristoyl-2-palmitoyl-sn-glycero 3-phosphocholine, 1-myristoyl-2-stearoyl-sn-glycero-3-phosphocholine, 1-palmitoyl-2-myristoyl-sn-glycero-3-phosphocholine, 1-palmitoyl-2-oleoyl-sn-glycero-3-phosphocholine, 1-palmitoyl-2-stearoyl-sn-glycero-3-phosphocholine, 1-stearoyl-2-myristoyl-sn-glycero-3-phosphocholine, 1-stearoyl-2-oleoyl-sn-glycero-3-phosphocholine, 1-stearoyl-2-palmitoyl-sn-glycero-3-phosphocholine; a phosphatidylethanolamine such as 1,2-ierucoyl-sn-glycero-3-phosphoethanolamine, 1,2-dilauroyl-sn-glycero-3-phosphoethanolamine, 1,2-dimyristoyl-sn-glycero-3-phosphoethanolamine, 1,2-dioleoyl-sn-glycero-3-phosphoethanolamine, 1,2-dipalmitoyl-sn-glycero-3-phosphoethanolamine, 1,2-distearoyl-sn-glycero-3-phosphoethanolamine, 1-palmitoyl-2-oleoyl-sn-glycero-3-phosphoethanolamine; a phosphatidylglycerol such as 1,2-dierucoyl-sn-glycero-3[phospho-rac-(1-glycerol . . . ) (sodium salt), 1,2-dilauroyl-sn-glycero-3[phospho-rac-(1-glycerol . . . ) (sodium salt), 1,2-dilauroyl-sn-glycero-3[phospho-rac-(1-glycerol . . . ) (ammonium salt), 1,2-dimyristoyl-sn-glycero-3[phospho-rac-(1-glycerol . . . ) (sodium salt), 1,2-dimyristoyl-sn-glycero-3[phospho-rac-(1-glycerol . . . ) (ammonium salt), 1,2-dimyristoyl-sn-glycero-3[phospho-rac-(1-glycerol . . . ) (sodium/ammonium salt), 1,2-dioleoyl-sn-glycero-3[phospho-rac-(1-glycerol . . . ) (sodium salt), 1,2-dipalmitoyl-sn-glycero-3[phospho-rac-(1-glycerol . . . ) (sodium salt), 1,2-dipalmitoyl-sn-glycero-3[phospho-rac-(1-glycerol . . . ) (ammonium salt), 1,2-distearoyl-sn-glycero-3[phospho-rac-(1-glycerol . . . ) (sodium salt), 1,2-distearoyl-sn-glycero-3[phospho-rac-(1-glycerol . . . ) (ammonium salt), 1-palmitoyl-2-oleoyl-sn-glycero-3[phospho-rac-(1-glycerol) . . . ] (sodium salt); or a phosphatidylserine such as 1,2-dilauroyl-sn-glycero-3-phosphoserine (sodium salt), 1,2-dimyristoyl-sn-glycero-3-phosphoserine (sodium salt), 1,2-dioleoyl-sn-glycero-3-phosphoserine (sodium salt), 1,2-dipalmitoyl-sn-glycero-3-phosphoserine (sodium salt), or 1,2-distearoyl-sn-glycero-3-phosphoserine (sodium salt).

5.4.3. Targeting Moieties

In some embodiments, one or both of the agents to be delivered and/or targeting moiety can be associated with the surface of, encapsulated within, surrounded by, and/or dispersed throughout the nanoparticle. In certain embodiments, the targeting moiety is found substantially only on the surface of the particle. In certain embodiments, targeted particles in accordance with the present invention comprise a targeting moiety which specifically binds to one or more targets associated with an organ, tissue, cell, extracellular matrix, extracellular compartment, and/or intracellular compartment. A targeting moiety may be a nucleic acid (e.g., aptamer), polypeptide (e.g., antibody), glycoprotein, small molecule, carbohydrate, lipid, a magnetic particle, etc. For a review, see Mishra et al., 2010 Nanomedicine 6:9-24, the content of which is hereby incorporated by reference in its entirety.

For example, a targeting moiety can be an aptamer, which is generally an oligonucleotide (e.g., DNA, RNA, or an analog or derivative thereof) that binds to a particular target, such as a polypeptide. In general, the targeting function of the aptamer is based on the three-dimensional structure of the aptamer and interactions beyond the traditional Watson-Crick base pairing of complementary strands. See Ellington & Szostak, 1990, Nature, 346, 818-822; U.S. Pat. Nos. 5,763,177, 6,001,577, 6,291,184, 6,458,539 (photoreactive aptamers); 5,683,867, 6,083,696 (aptamers with non-nucleic acids, peptides); 5,705,337 (covalent linkage methods), all of which are hereby incorporated by reference in their entirety. In some embodiments, the aptamer is a spiegelmer. See Vater & Klussmann, 2003, Curr Opin Drug Discov Devel 6, 253-261; U.S. Pat. No. 7,629,456; and U.S. Appn. Pub. No. 2009/0192100, the contents of which are hereby incorporated by reference in their entirety. In some embodiments, the targeting moiety is a polypeptide (e.g., an antibody that specifically recognizes a tumor marker). In certain embodiments, the targeting moiety is an antibody or a fragment thereof. In certain embodiments, the targeting moiety is an Fc fragment of an antibody.

In some embodiments, the targeting moiety may bind to a marker that is exclusively or primarily associated with one or a few tissue types, with one or a few cell types, with one or a few diseases, and/or with one or a few developmental stages. In some embodiments, a target can comprise a protein (e.g., cell surface receptor, transmembrane protein, glycoprotein, etc.), a carbohydrate (e.g., glycan moiety, glycocalyx, etc.), a lipid (e.g., steroid, phospholipid, etc.), and/or a nucleic acid (e.g., DNA, RNA, etc.). In one embodiment, the targeting moiety is a folate or a folate analogue or derivative. In other embodiments, the targeting moity is an affinity ligand for the transferrin receptor or αVβ3 integrin such as an RGD (Arg-Gly-Asp) peptide, see Lin 2005 Nanomedicine 1:110-114, the content of which is hereby incorporated by reference in its entirety. In another embodiment, the targeting moity is an antibody specific for a particular cell surface molecule such as platelet/endothelial cell adhesion molecule 1 (PECAM-1), Shuvaev, Muzykantov et al., 2011 FASEB J 25(1):348-357; or intercellular adhesion molecule 1 (ICAM-1), Calderon, Muzykantov et al., 2010 J Control Release doi:10.1016 /j.jconrel.2010.10.025; or Simone Muzykantov et al., 2010 Meth Mol Bio 610 145-164, the contents of which are hereby incorporated by reference in their entirety.

In some embodiments, the targeting moiety is a peptide binder developed using phage display technology. See Smith & Petrenko, 1997, Chem Rev 97 (2): 391-410; Kehoe & Kay, 2005 Chem Rev 105 (11): 4056-4072; Lunder et al., 2005 Appl Biochem Biotechnol 127 (2): 125-31; McCafferty et al., 1990 Nature 348: 552-554; U.S. Pat. Nos. 5,427,908 and 5,580,717 (Dower); 5,885,793 (Griffiths); 6,248,516 and 6,545,142 (Winter); 6,291,158 and 6,291,159 (Winter, Lerner); 7,118,879 (Ladner); 7,803,913 (Dimitrov & Zhang), the contents of which are hereby incorporated by reference in their entirety.

In some embodiments, the targeting moiety is a moiety that specifically binds a marker that is present exclusively or in higher amounts on a malignant cell, e.g., a tumor antigen. In one embodiment, the targeting moiety is an antibody specific for the EGF receptor (EGFR). In some embodiments, a marker is a prostate cancer marker. In certain embodiments, the prostate cancer marker is prostate specific membrane antigen (PSMA), a 100 kDa transmembrane glycoprotein that is expressed in most prostatic tissues, but is more highly expressed in prostatic cancer tissue than in normal tissue. In some embodiments, a marker is a breast cancer marker. Examples of a breast cancer specific targeting moities may be found in Steinhauser et al., 2006, Biomaterials 27:4975-4983 (HER-2 receptor) or Torchilin 2007 AAPS J 9:128-147 (vasoactive intestinal peptide (VIP)), the contents of which are hereby incorporated by reference in their entirety. In some embodiments, a marker is a colon cancer marker. In some embodiments, a marker is a rectal cancer marker. In some embodiments, a marker is a lung cancer marker. In some embodiments, a marker is a pancreatic cancer marker. In some embodiments, a marker is an ovarian cancer marker. In some embodiments, a marker is a bone cancer marker. In some embodiments, a marker is a renal cancer marker. In some embodiments, a marker is a liver cancer marker. In some embodiments, a marker is a neurological cancer marker. In some embodiments, a marker is a gastric cancer marker. In some embodiments, a marker is a testicular cancer marker. In some embodiments, a marker is a head and neck cancer marker. In some embodiments, a marker is an esophageal cancer marker. In some embodiments, a marker is a cervical cancer marker.

In some embodiments, the targeting moiety is a magnetic particle. For examples of magnetic particles, see Cheng et al., 2005 Pharm Res 25(3) 557-564; Arruebo et al., 2007 Nano Today 2:22-32; Jain 2008 Biomaterials 29:4012-4021; U.S. Pat. Nos. 5,427,767 and 6,576,221 (Kresse et al.), the contents of which are hereby incorporated by reference in their entirety.

5.4.4. Additional Agents in Combination

According to the present invention, any agents can be co-delivered with the DNA double-stranded break (DSB) repair enzyme inhibitor, including, for example, chemotherapeutic agents (e.g., anti-cancer agents), diagnostic agents (e.g., contrast agents; radionuclides; and fluorescent, luminescent, and magnetic moieties), prophylactic agents (e.g., vaccines), and/or nutraceutical agents (e.g., vitamins, minerals, etc.) may be delivered in combination with an agent that includes a radioisotope (e.g., a radiotherapeutic or radiodiagnostic agent). Exemplary agents to be delivered in accordance with the present invention include, but are not limited to, small molecules (e.g., cytotoxic agents), nucleic acids (e.g., RNAi agents), proteins (e.g., antibodies), lipids, carbohydrates, hormones, metals, radioactive elements and compounds, drugs, vaccines, immunological agents, etc., and/or combinations thereof. In some embodiments, the agents to be delivered are agents useful in the treatment of cancer (e.g., cervical, esophageal, gastric, head and neck, lung, pancreatic, prostate, or rectal cancer). In some embodiments, the non-radioactive agent to be delivered may be a mixture of non-radioactive agents. In certain particular embodiments, the chemotherapeutic agent to be delivered is a combination of chemotherapeutic agents.

5.4.5. Imaging Agents

For in vivo imaging purposes, the nanoparticles may be labeled with a positron-emitting isotope (e.g., 18F) for positron emission tomography (PET), gamma-ray isotope (e.g., 99mTc) for single photon emission computed tomography (SPECT), a paramagnetic molecule or nanoparticle (e.g., Gd³⁺ chelate or coated magnetite nanoparticle) for magnetic resonance imaging (MRI), a near-infrared fluorophore for near-infra red (near-IR) imaging, a luciferase (firefly, bacterial, or coelenterate), green fluorescent protein, or other luminescent molecule for bioluminescence imaging, or a perfluorocarbon-filled vesicle for ultrasound.

Furthermore, such reagents may include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are described, for example, in U.S. Pat. Nos. 4,452,720 (Harada et al.); 5,227,487 (Haugland and Whitaker); and 5,543,295 (Bronstein et al.). Other fluorescent labels suitable for use in the practice of this invention include a fluorescein dye. Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. Nos. 4,439,356 (Khanna and Colvin); 5,066,580 (Lee), 5,750,409 (Hermann et al.); and 6,008,379 (Benson et al.). The nanoparticles may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®, and other rhodamine dyes. Other rhodamine dyes can be found, for example, in U.S. Pat. Nos. 5,936,087 (Benson et al.), 6,025,505 (Lee et al.); 6,080,852 (Lee et al.). The nanoparticles may include a cyanine dye, such as, for example, Cy3, Cy3B, Cy3.5, Cy5, Cy5.5, Cy7. Phosphorescent compounds including porphyrins, phthalocyanines, polyaromatic compounds such as pyrenes, anthracenes and acenaphthenes, and so forth, may also be used.

5.4.6. Nanoparticle Fabrication

In another aspect, the invention provides methods of preparing the nanoparticles. The nanoparticles may be manufactured using any available method which does not interfere with the therapeutic, diagnostic, prophylactic, or other biological properties of the inventive particle. In certain embodiments, nanoprecipitation is used to form the nanoparticles (see Section 6.1). A lipid monolayer may then be self-assembled on the core polymeric nanoparticle. As would be appreciated by those of skill in the art, other techniques for preparing particles may also be used, for example, spray drying, double emulsion, single emulsion, etc. In certain embodiments, the particles are prepared by nanoprecipitation using microfluidic devices.

Alternative, methods of precipitation by slow addition of a polymer and drug dissolved in an organic solvent to an aqueous solution have been described. See Cheng et al., 2007 Biomaterials 28:869-879; or Brodbeck et al., 1999, Pharm Res 16:1825, the contents of which are hereby incorporated by reference in their entirety. In other embodiments, the nanoparticles may be prepared by an emulsion-based encapsulation process. The emulsion-based encapsulation may utilize an interfacial polymerization process. The nanoparticles in the emulsion may be recovered by a variety of means. In one embodiment, the nanoparticles are recovered by precipitation by changing the temperature, pH, or salt concentration. In another embodiment, the nanoparticles are recovered when the solvent evaporated. In another embodiment, the encapsulation may utilize a spray dry encapsulation process where the desired polymer and wortmannin are dissolved in a solvent and spray atomized See Palmieri et al., 2001, Drug Dev Ind Pharm 27: 195, the content of which is hereby incorporated by reference in its entirety. In another embodiment, the nanoparticles are prepared using a spray-freeze methodology where the polymer and wortmannin are plunged into liquid nitrogen (Johnson et al., 1997, Pharm Res 14:730, the content of which is hereby incorporated by reference in its entirety).

Alternatively, the nanoparticles may be prepared by mechanical means with a high shear mill, a homogenizer, a ball mill, or a stirred-media mill. In some embodiments, the particles are spheres, spheroids, flat, plate-shaped, cubes, cuboids, ovals, ellipses, cylinders, cones, or pyramids. In another embodiment, nanoparticles may have a variety of shapes, such as an elongated ellipsoid, a ribbon, or a tubule, see Simone, Muzykantov et al., 2008 Exp Opin Drug Deliv 5(12):1282-1300, the content of which is hereby incorporated by reference in its entirety. Additional embodiments include, without limitation, methods to prepare nanoparticles with highly controlled geometry such as the nanoparticles are prepared using a fluoropolymer nanofabrication technique. See Gratton, Desimone et al., 2008, Accts Chem Res 41(12) 1685-1695; WO 2008/106503 (Desimone et al.); WO 2007/024323 (Desimone et al.); WO 2005/101466 (Desimone et al.), the contents of which are hereby incorporated by reference in their entirety.

Association of the wortmannin to be delivered and/or the targeting moiety with the particle can be achieved in a variety of different ways. Physical association may be covalent or non-covalent. A covalent association may or may not involve a linker moiety. The particle, targeting moiety, and/or one or both agents to be delivered may be directly associated with one another, e.g., by one or more covalent bonds, or the association may be mediated by one or more linkers. In some embodiments, a linker is a cleavable linker. In some embodiments, a linker is an aliphatic or heteroaliphatic linker. In some embodiments, the linker is a polyalkyl linker. In certain embodiments, the linker is a polyether linker. In certain embodiments, the linker is a polyethylene linker. In certain specific embodiments, the linker is a polyethylene glycol (PEG) linker. For example, the chelator may be associated with the polymer of the particle through a PEG linker. In some embodiments, targeted particles in accordance with the present invention may be used to treat, alleviate, ameliorate, relieve, delay onset of, inhibit progression of, reduce severity of, and/or reduce incidence of one or more symptoms or features of a disease, disorder, and/or condition. In some embodiments, inventive targeted particles may be used to treat cancer. In certain embodiments, inventive targeted particles may be used to treat a benign neoplasm. In certain embodiments, inventive targeted particles may be used to treat an inflammatory disease. In certain embodiments, inventive targeted particles may be used to treat an infectious disease. In certain embodiments, inventive targeted particles may be used to treat a cardiovascular disease (e.g., atherosclerosis). The compositions, according to the method of the present invention, may be administered using any amount and any route of administration effective for treatment.

The system may be further modified for selectively delivering DNA double-stranded break (DSB) repair enzyme inhibitors such as wortmannin in combination with other agents to particular organs, tissues, cells, and/or intracellular compartments. The targeting of the drug delivery device would allow for the targeted delivery of both wortmannin and similar PI3Ks inhibitors and other chemotherapeutic agents and the radiotherapeutic agent in one drug delivery device. In one aspect, the drug delivery system is a particle (e.g., picoparticle, nanoparticle, or microparticle) comprising a therapeutic, diagnostic, or prophylactic agent, and a radiotherapeutic or radiodiagnostic agent. In certain embodiments, the particle is a polymeric particle. In certain embodiments, the particle comprises a polymeric core with a shell coating the core. In certain embodiments, the particle comprises a polymeric core coated with a lipid (e.g., a lipid monolayer or lipid bilayer). In certain embodiments, the particle is a liposome. In certain embodiments, the particle is a micelle. One or both of the agents to be delivered may be inside the particle (e.g., in the core), in the shell or coating portion of the particle, or associated with the surface of the particle.

### 5.5. Pharmaceutically Acceptable Compositions

Provided herein are pharmaceutical compositions comprising a compound provided herein, e.g., a nanoparticle containing DNA double-stranded break (DSB) repair enzyme inhibitors such as wortmannin or a wortmannin analogue, as an active ingredient, or a pharmaceutically acceptable salt, solvate or hydrate thereof in combination with a pharmaceutically acceptable vehicle, carrier, diluent, or excipient, or a mixture thereof.

The compound provided herein may be administered alone, or in combination with one or more other compounds provided herein. The pharmaceutical compositions that comprise a compound provided herein, e.g., nanoparticle containing DNA double-stranded break (DSB) repair enzyme inhibitors such as wortmannin or a wortmannin analogue, can be formulated in various dosage forms for oral, parenteral, and topical administration. The pharmaceutical compositions can also be formulated as modified release dosage forms, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, 21st Ed., Lippincott, Williams & Wilkins, Baltimore, Md., 2006; Modified-Release Drug Delivery Technology, Rathbone et al., Eds., Drugs and the Pharmaceutical Science, Marcel Dekker, Inc.: New York, N.Y., 2003; Vol. 126).

In one embodiment, the pharmaceutical compositions are provided in a dosage form for oral administration, which comprise a compound provided herein, e.g., nanoparticle containing DNA double-stranded break (DSB) repair enzyme inhibitors such as wortmannin or a wortmannin analogue, or a pharmaceutically acceptable salt, solvate or hydrate thereof; and one or more pharmaceutically acceptable excipients or carriers.

In another embodiment, the pharmaceutical compositions are provided in a dosage form for parenteral administration, which comprise a compound provided herein, e.g., nanoparticle containing DNA double-stranded break (DSB) repair enzyme inhibitors such as wortmannin or a wortmannin analogue, or a pharmaceutically acceptable salt, solvate or hydrate thereof and one or more pharmaceutically acceptable excipients or carriers.

In yet another embodiment, the pharmaceutical compositions are provided in a dosage form for topical administration, which comprise a compound provided herein, e.g., nanoparticle containing DNA double-stranded break (DSB) repair enzyme inhibitors such as wortmannin or a wortmannin analogue, or a pharmaceutically acceptable salt, solvate or hydrate thereof; and one or more pharmaceutically acceptable excipients or carriers.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons. The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In one embodiment, the therapeutically effective dose is from about 0.1 mg to about 2,000 mg per day of a compound provided herein. The pharmaceutical compositions therefore should provide a dosage of from about 0.1 mg to about 2000 mg of the compound. In certain embodiments, pharmaceutical dosage unit forms are prepared to provide from about 1 mg to about 2000 mg, from about 10 mg to about 1000 mg, from about 20 mg to about 500 mg or from about 25 mg to about 250 mg of the essential active ingredient or a combination of essential ingredients per dosage unit form. In certain embodiments, the pharmaceutical dosage unit forms are prepared to provide about 10 mg, 20 mg, 25 mg, 50 mg, 100 mg, 250 mg, 500 mg, 1000 mg or 2000 mg of the essential active ingredient.

5.5.1. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfate and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including a-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein can be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In one embodiment, the lyophilized nanoparticles are provided in a vial for reconstitution with a sterile aqueous solution just prior to injection. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions. The pharmaceutical compositions provided herein can be formulated as immediate or modified release dosage forms, including delayed-, sustained, pulsed-, controlled, targeted-, and programmed-release forms.

The pharmaceutical compositions can be formulated as a suspension, solid, semi-solid, or thixotropic liquid, for administration as an implanted depot.

5.5.2. Oral Administration Compositions

The pharmaceutical compositions provided herein can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, troches, lozenges, pastilles, cachets, pellets, medicated chewing gum, bulk powders, effervescent or non-effervescent powders or granules, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W. R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Hydrophilic polymer formulations have been widely used for improved oral availability such as ethylene oxides, hydroxy propyl methyl cellulose (HPC), poly(ethylene oxide) (PEO), polyvinyl alcohol (PVA), poly(hydroxyethylmethyl acrylate) methyl methacrylate (PHEMA), or vinyl acetate (PCT Pub. No. WO1999/37302 (Alvarez et al.); Dimitrov & Lambov, 1999, Int J Pharm 189 105-111; Zhang et al., 1990, Proc Int. Symp Controlled Release Bioact. Mater. 17, 333, the contents of which are hereby incorporated by reference in their entirety). Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545, the contents of which are hereby incorporated by reference in their entirety. The capsules may also be coated as known by those of skill in the art in order to modify or sustain dissolution of the active ingredient.

The pharmaceutical compositions provided herein can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfate, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458, the content of which is hereby incorporated by reference in its entirety.

The pharmaceutical compositions provided herein can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms. The pharmaceutical compositions provided herein can be formulated as immediate or modified release dosage forms, including delayed-, sustained, pulsed-, controlled, targeted-, and programmed-release forms.

The pharmaceutical compositions provided herein can be co-formulated with other active ingredients which do not impair the desired therapeutic action, or with substances that supplement the desired action.

5.5.3. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator can be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions provided herein for inhaled/intranasal administration can further comprise a suitable flavor, such as menthol and levomenthol, or sweeteners, such as saccharin or saccharin sodium.

The pharmaceutical compositions provided herein for topical administration can be formulated to be immediate release or modified release, including delayed-, sustained-, pulsed-, controlled-, targeted, and programmed release.

### 5.6. Modified Release Formulations

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500, the contents of which are hereby incorporated by reference in their entirety.

5.6.1. Matrix Controlled Release Devices

The pharmaceutical compositions provided herein in a modified release dosage form can be fabricated using a matrix controlled release device known to those skilled in the art (see, Takada et al. in “Encyclopedia of Controlled Drug Delivery,” Vol. 2, Mathiowitz Ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethylene terephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed; the polymer viscosity; the particle sizes of the polymer and/or the active ingredient(s); the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, melt-granulation followed by compression.

5.6.2. Osmotic Controlled Release Devices

The pharmaceutical compositions provided herein in a modified release dosage form can be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220, the contents of which are hereby incorporated by reference in their entirety.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27, the contents of which are hereby incorporated by reference in their entirety.

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918, the contents of which are hereby incorporated by reference in their entirety. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

5.6.3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 5.7. Enhanced Radiation Treatments

The invention also provides methods of enhanced radiation treatment by concurrent administration of the nanoparticles described herein. The enhanced treatments may be neoadjuvant, primary or adjuvant chemoradiotherapy. A chemoradiation protocol may involve a single dose of radiation of 10 to 20 Gy and the maximum tolerated dose of a DNA double-stranded break (DSB) repair enzyme inhibitor such as wortmannin in nanoparticles. Alternatively, a fractional radiation may be used such as a radiation dose of 50 Gy (Gray) to 80 Gy in 1.8 Gy to 2.0 fractions and a maximum tolerated dose of DNA double-stranded break (DSB) repair enzyme inhibitor nanoparticles given twice a week. Alternatively, the radiation dose may be 30 to 40 Gy in 2 to 3 Gy fractions with weekly treatments of DNA double-stranded break (DSB) repair enzyme inhibitor nanoparticles.

The DNA double-stranded break (DSB) repair enzyme inhibitor nanoparticles may also be used for chemoradiation in combination with existing chemoradiation agents. Non-limiting examples of such combinations for specific cancers are Anal cancer, 5-FU or mitomycin C; Bladder cancer, clsplatin; Cervical cancer, cisplatin, 5-FU, or hydroxyurea; Cholangiocarcinoma, 5-FU; Esophageal cancer, cisplatin/5-FU; Gastric cancer, cisplatin or 5-FU; Glioblastoma, temozolomide; Head and neck cancer, cisplatin, 5-FU, 5-FU and hydroxyurea, or cetuximab; Non-small-cell lung cancer, cisplatin, carboplatin/paclitaxel, or cisplatin/etoposide; Pancreatic cancer 5-FU; Rectal cancer, 5-FU; Sarcoma doxorubicin; Small-cell lung cancer Cisplatin/etoposide. See Seiwert et al., 2007 Nature Clin Prac Oncol 4(2) 86-100 and Morgan et al., 2008 Clin Cancer Res 14(21): 6744-6750, the contents of which are hereby incorporated by reference in their entirety.

The following Examples further illustrate the invention and are not intended to limit the scope of the invention.

### 6. EXAMPLES

### 6.1. Materials and Methods

Wortmannin was obtained from Sigma-Aldrich (St. Louis, Mo.). PLGA (poly(D,L-lactide-co-glycolide)) with a 50:50 monomer ratio, ester-terminated, and viscosity of 0.72-0.92 dl/g was purchased from Durect Corporation (Pelham, Ala.). Soybean lecithin consisting of 90-95% phosphatidylcholine was obtained from MP Biomedicals (Solon, Ohio). DSPE-PEG2000-COOH (1,2-distearoyl-sn-glycero-3-phosphoethanolamine-N-carboxy(polyethylene glycol) 2000) and DSPE-PEG2000-Folate (1,2-distearoyl-sn-glycero-3-phosphoethanolamine-N-carboxy(polyethylene glycol) 2000 Folate) was obtained from Avanti Polar Lipids (Alabaster, Ala.). KB, PC3 and HT 29 cell lines were obtained from ATCC (Manassas, Va.). CellTiter 96® Non-Radioactive cell proliferation assay (MTS) was purchased from Promega Corporation (Madison, Wis.).

Formulation and Characterization of NP Wortmannin:

The nanoparticles were synthesized from PLGA, soybean lecithin and DSPE-PEG-COOH using a previously reported nanoprecipitation technique (Chan et al. 2009, Biomaterials, 30, 1627-1634, the contents of which is hereby incorporated by reference in their entirety). Briefly, PLGA and wortmannin was dissolved in acetonitrile at a concentration of 10 and 1 mg/ml respectively. Wortmannin dissolved in acetonitrile was mixed with PLGA in acetonitrile at a ratio of 20% wt/wt PLGA/wortmannin. Lecithin, DSPE-PEG-COOH and DSPE-PEG-Folate (for targeted NPs) (7:2.4:0.6, molar ratio) were dissolved in 1.85 ml 4% ethanol aqueous solution at 15% of the PLGA polymer weight and heated to 55° C. The PLGA/wortmannin acetonitrile solution was then added dropwise to the heated aqueous solution under gentle stirring followed by 3 minutes of vortexing. The nanoparticles were allowed to self-assemble for 2 h with continuous stirring under vacuum. The NP solution was washed with PBS using an Amicon Ultra-4 centrifugal filter (Millipore, Billerica, Mass.) with a molecular weight cut-off of 30 kDa and then resuspended in an equal volume of 1×PBS to obtain a final desired concentration. The NPs were used immediately. NP size (diameter, m) and surface charge (ζ-potential, mV) were obtained with a ZetaPALS dynamic light scattering detector (Brookhaven Instruments Corporation, Holtsville, N.Y.). Transmission electron microscopy (TEM) images were obtained at the Microscopy Services Laboratory Core Facility at the UNC School of Medicine. A TEM image of the wortmannin nanoparticles showed that the particles were monodispersed with well-defined spherical shape and an average size of 40 nm.

Nanoparticle Wortmannin Release:

To measure the drug loading yield and release profile of wortmannin, 2 mL NP solutions at a concentration of 1 mg/mL were split equally into 20 Slide-ALyzer MINI dialysis microtubes with a molecular weight cut-off of 10 kDa (Pierce, Rockford, Ill.) and subject to dialysis against 4 L phosphate buffer saline (PBS) with gentle stirring at 37° C. PBS was changed periodically during the dialysis process. At the indicated times, 0.1 mL of solution from two microtubes was removed and mixed with an equal volume of acetonitrile to dissolve the NPs. Wortmannin content was subjected to quantitative analysis using an Agilent 1100 HPLC (Palo Alto, Calif.) equipped with a C18 chromolith flash column (Merck KGaA Darmstadt, Germany). Wortmannin absorbance was measured by a UV-VIS detector at 254 nm and a retention time of 1.6 min in 0.25 mL/min 50:50 acetonitrile/water non-gradient mobile phase.

Clonogenic Survival Assay:

Cells were seeded at various densities ranging from 100 to 20000 cells in 5 ml of culture medium in 25 ml flasks 1 day prior to treatment. Cells were treated with different wortmannin delivery modalities for 3 hours. Post incubation, the cells were washed 2 times with sterile PBS trypsinised, and plated with fresh media. The cells were then radiated at 0, 2, 4, 6, and 8 Gy respectively. The cells were further incubated for 12 days after irradiation. After 12 days, the cells were fixed in 50% Acetone/50% Methanol and stained with trypan blue. All colonies with over 50 cells were counted. The relative cell surviving fraction was calculated by dividing the number of colonies of radiated cells by that of control.

Inhibition of DNA-PK Phosphorylation with Wortmannin Nanoparticle:

M059K glioblastoma cells were pre-treated with 25 μM wortmannin or wortmannin nanoparticle (which contains an equivalent amount of wortmannin) for 3 h and subsequently irradiated (4 Gy). There were two control groups: cells irradiated with 4 Gy (no wortmannin treatment) and cells with no radiation and no wortmannin treatment. Cell lysates were collected in HNTG buffer at the indicated time points post irradiation. The protein concentration was measured by BCA protein assay (Pierce, Rockford, Ill.). Proteins were separated by electrophoresis on 4-15% gradient SDS-polyacrylamide gels and transferred to a PVDF membrance (Millipore, Billerica, Mass.). The membranes were blocked in TBST containing 5% wt/vol albumin from bovine serum, Cohn V fraction for 2 h. Primary antibodies used: DNA PKcs (Abcam, Cambridge, Mass.) DNA-PKcs (phospho 52056) (Abcam) and β-Actin (13E5) (Cell Signaling) at dilutions indicated by the manufacturers. Secondary antibodies were α-mouse IgG HRP linked antibody (Cell Signaling) or α-rabbit IgG HRP linked antibody (Cell Signaling)

Animals:

All the mice were obtained from Charles River and housed in an AALAC accredited facility in sterile housing following all IACUC requirements. Mice were housed in filter top cages and provided with sterile food and water.

Maximum Tolerated Dose (MTD):

MTD, defined as the highest possible dose resulting in no animal deaths and less than 20% weight loss was evaluated for wortmannin formulations. Non-tumor-bearing mice (NOD/SCID, C57BL/6, CB6F1 and CD1) were injected with varying amounts of wortmannin nanoparticle and wortmannin in cremophor normalized to mg wortmannin injected/kg of mice body weight. The animals were weighed and observed daily for any change in physical activity. Weight loss greater than 20% and/or lethargy were interpreted as indications of toxicity and the mice were immediately euthanized.

Table 2 compares the maximum tolerated dose (MTD) of wortmannin in cremophor and wortmannin nanoparticle in different mouse strains.

TABLE 2

| | Wortmannin | Wortmannin Nanoparticle |
|---|---|---|
| Mouse Strain | MTD (mg/kg) | MTD (mg/kg) |
| NOD/SCID | 0.7 | 2.2 [110] |
| C57BL/6 (Black) | 0.7 | 2.4 [120] |
| CB6F1 (Brown) | 0.5 | 2.5 [125] |
| CD1 (White) | 0.7 | 2.4 [120] |

Table 2: MTD (Maximum Tolerated Dose) of Wtmn formulations in four different strains of mice. Numbers represent mg/kg of wortmannin, either in cremophor solution or encapsulated in nanoparticle (2% w/w), to reach MTD. Numbers in brackets [ ] represent the mg/kg of the wortmannin nanoparticle including the weight of the nanoparticle.

Toxicity:

CD1 mice were given tail vein i.v. injection with different wortmannin formulations (wortmannin nanoparticle or wortmannin in cremophor) at the indicated doses (⅕^{th} the MTD or 1/10^{th} the MTD). After 24 h, submandibular bleeding was performed. Blood (100-200 ul) was collected in heparin coated tubes to prevent coagulation. Whole blood samples were immediately submitted for blood toxicity to the Animal Clinical Laboratory Core Facility at UNC. For liver toxicity studies, blood samples were centrifuged (15000×g for 10 minutes) to separate blood cells from the plasma and samples were submitted for analysis.

Table 3 and 4 show the toxicity profile in mice of wortmannin in cremophor and wortmannin nanoparticle.

TABLE 3
⅕th MTD of Wtmn (0.14 mg/kg)

| | | Normal | | |
|---|---|---|---|---|
| | | range | Wtmn | NP Wtmn |
| Hematologic | WBC | 3.5-10 | 2.8 ± 0.28 | 5.1 ± 0.42 |
| Toxicity | | (103/μl) | | |
| | Granulocytes | 1.2-8 | 0.8 ± 0.14 | 2.9 ± 0.07 |
| | | (103/μl) | | |
| | Lymphocytes | 0.5-5 | 0.4 ± 0.42 | 3.56 ± 0.21 |
| | | (103/μl) | | |
| | Monocytes | 0.1-1.5 | 0.55 ± 0.07 | 0.7 ± 0.14 |
| | | (103/μl) | | |
| Hepatotoxicity | ALT U/L | 40-50 U/L | 134 ± 17 | 82 ± 12 |
| | AST U/L | 40-50 U/L | 44 ± 3 | 46 ± 7 |

TABLE 4
1/10th MTD of Wtmn (0.07 mg/kg)

| | | Normal | | |
|---|---|---|---|---|
| | | range | Wtmn | NP Wtmn |
| Hematologic | WBC | 3.5-10 | 4.3 ± 0.13 | 4.9 ± 0.20 |
| Toxicity | | (103/μl) | | |
| | Granulocytes | 1.2-8 | 2.4 ± 0.11 | 3.7 ± 0.12 |
| | | (103/μl) | | |
| | Lymphocytes | 0.5-5 | 0.9 ± 0.28 | 1.05 ± 0.21 |
| | | (103/μl) | | |
| | Monocytes | 0.1-1.5 | 0.8 ± 0.42 | 0.5 ± 0.14 |
| | | (103/μl) | | |
| Hepatotoxicity | ALT U/L | 40-50 U/L | 64 ± 11 | 60 ± 9 |
| | AST U/L | 40-50 U/L | 40 ± 7 | 42 ± 10 |

Table 3 and 4. Toxicity profile in CD1 mice post i.v. injection of wortmannin formulations. Mice were treated with indicated doses of wortmannin or wortmannin nanoparticle containing an equivalent dose of wortmannin (2% w/w). (Single tail vein i.v. injection, toxicity profile 3 d post injection).

Tumor Implantation:

To induce the tumor, 200 uL of matrigel and serum free media containing 1×10⁶ KB cells were subcutaneously injected into the right flank of the mice. Tumor growth and body weight were recorded daily.

Excised Organ Biodistribution:

Six- to eight-week old tumor bearing NOD/SCIDS mice were administered via tail vain injection 0.2 mL suspension of 111In labeled nanoparticles (200 micrograms total nanoparticles/mouse), and were sacrificed at two, eight and twenty-four hours post-injection by CO₂ intoxication for dissection. Three to four mice were used per data point. Blood was collected via cardiac puncture. Organs and tissues were washed in PBS, weighed, and then counted in a Perkin Elmer 2470 Automatic Gamma Counter (Perkin Elmer, Waltham, Mass.)) using a 15-550 KeV window. Results were expressed as percentage of total injected radioactivity in each organ divided by the organ mass (% ID/g).

Anti-Tumor Efficacy Studies:

One week after cells injection and when the volume of the tumor was around 100 mm³, animals were randomly divided into six main groups: No treatment—control group receiving no treatment of any sort, XRT-mice only treated with XRT, Free Wort—mice treated with a single dose of wortmannin in cremophor at 1/10^{th} the MTD, Free Wort+XRT—mice treated with a single dose of wortmannin in cremophor at 1/10^{th} the MTD and treated with XRT, Wort NP—mice treated with wortmannin nanoparticle at 1/10^{th} the MTD of free wortmannin, and Wort NP+XRT—mice treated with wortmannin nanoparticle at 1/10^{th} the MTD of free wortmannin and treated with XRT. Any mice that received XRT were irradiated 3 hours post injection with a single dose of 6 Gy. Tumor sizes and weights were monitored daily. The tumor volume was calculated with formula

Volume=0.5×a×b2

‘a’—larger diameter of tumor, ‘b’ smaller diameter of tumor.

Wortmannin as a Chemosensitizer:

H460 Cells were plated on 96 well plates at a density of 20,000 cells per well. Free wortmannin was added to cells in a total final volume of 0.1 mL per well, and were incubated for 6 hours. At the end of incubation, cells were gently washed twice with sterile PBS, and were further incubated with either medium (control), 0.5 uM Etoposide or 10 uM Irinotecan for the period of one doubling time (24 h) at 37 C and 5% CO2. To evaluate cell viability, MTS assay was performed according to manufacturer's instructions.

KU55933 Nanoparticle:

KU55933 was loaded in a nanoparticle at 3% w/v

KU55933 Nanoparticle as a Radiosensitizer:

A549 cells were seeded at various densities ranging from 100 to 20000 cells in 5 ml of culture medium in 25 ml flasks 1 day prior to treatment. Cells were either not treated or treated with KU55933 nanoparticle (containing the equivalent of 10 uM KU55933) for 3 hours. Post incubation, the cells were washed 2 times with sterile PBS trypsinised, and plated with fresh media. The cells were then radiated at 0, 2, 4, 6, and 8 Gy respectively. The cells were further incubated for 12 days after irradiation. After 12 days, the cells were fixed in 50% Acetone/50% Methanol and stained with trypan blue. All colonies with over 50 cells were counted. The relative cell surviving fraction was calculated by dividing the number of colonies of radiated cells by that of control.

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

1. A nanoparticle comprising (i) a polylactide polyglycolide (PLGA) copolymer, (ii) a polyethylene glycol (PEG), and (iii) a wortmannin or a wortmannin analogue.

2. (canceled)

3. The nanoparticle of claim 1, wherein the polylactide polyglycolide (PLGA) copolymer is a poly(lactide-co-glycolide) copolymer with a lactic acid to glycolic acid monomer ratio from about 10:90 to about 90:10.

4. The nanoparticle of claim 1, further comprising a lipid.

5. The nanoparticle of claim 4, consisting essentially of (i) the polylactide polyglycolide (PLGA) copolymer, (ii) the polyethylene glycol (PEG), (iii) the wortmannin or a wortmannin analogue, and the lipid.

6. The nanoparticle of claim 4, wherein the polylactide (PLA) is a polylactide polyglycolide (PLGA) copolymer, the polylactide is coated with the lipid, and the lipid is coated with the polyethylene glycol (PEG).

7. The nanoparticle of claim 1, wherein the wortmannin or wortmannin analogue is homogenously dissolved in the polylactide polyglycolide (PLGA) copolymer.

8. The nanoparticle of claim 1, further comprising a targeting moiety.

9. The nanoparticle of claim 8, wherein the targeting moiety is a carbohydrate, a fatty acid, a glycopeptide, a glycoprotein, a lipid, a peptide, a polymer, a polynucleotide, a protein, or a small molecule.

10. The nanoparticle of claim 9, wherein the small molecule is a folic acid analogue.

11. The nanoparticle of claim 9, wherein the protein is an antibody or an antibody fragment.

12. The nanoparticle of claim 9, wherein the polynucleotide is an aptamer.

13. (canceled)

14. The nanoparticle of claim 13, wherein the nanoparticle is about 10 nm to about 500 nm in at least one dimension.

15. The nanoparticle of claim 14, wherein the nanoparticle is about 20 nm to about 100 nm in at least one dimension.

16. The nanoparticle of claim 15, wherein the nanoparticle is about 50 nm to about 80 nm in at least one dimension.

17. The nanoparticle of claim 1, wherein the nanoparticle has a zeta potential between about −0 mV and about −50 mV.

18. The nanoparticle of claim 17, wherein the nanoparticle has a zeta potential between about −35 mV and about −45 mV.

19. The nanoparticle of claim 1, wherein the nanoparticle further comprises an imaging agent.

20. The nanoparticle of claim 19, wherein the imaging agent is a chemiluminescent agent, a colorimetric agent, an enzyme, a fluorophore, a light emitting agent, a light scattering agent, a magnetic resonance imaging (MRI) contrast agent, a positron emission tomography (PET) imaging agent, a radioisotope, or an ultrasound imaging agent.

21. The nanoparticle of claim 1, wherein the nanoparticle comprises about 1% to about 50% wortmannin or a wortmannin analogue by weight.

22. The nanoparticle of claim 1, wherein the nanoparticle comprises about 4% to about 30% wortmannin or a wortmannin analogue by weight.

23-31. (canceled)
